(19) United States
(12) Patent Application Publication
(10) Pub. No.: US 2019/0293564 A1
(43) Pub. Date: Sep. 26, 2019

(54) Network-Based Verification Systems and Methods

(71) Applicant: Oak Analytics Inc., Agoura Hills, CA (US)

(72) Inventors: Deepak Mehrotra, Thousand Oaks, CA (US); Steve K. Chen, Pacific Palisades, CA (US)

(73) Assignee: Oak Analytics Inc., Agoura Hills, CA (US)

(21) Appl. No.: 16/362,597

(22) Filed: Mar. 22, 2019

Related U.S. Application Data
(60) Provisional application No. 62/646,693, filed on Mar. 22, 2018.

(51) Int. Cl. G01N 21/65; G06N 20/00; H04L 29/06; G06Q 20/10; G01N 21/84

(52) U.S. Cl. CPC G01N 21/65; G06N 20/00; H04L 63/08; G01N 33/146; G01N 21/84; G01N 2201/129; G06Q 20/102

(57) ABSTRACT

Verification systems for testing food products or other samples include a mobile analytical device, a mobile accessory device such as a smart phone, and a remote, e.g., cloud-based, computing system. The mobile analytical device is adapted to generate a sensor output that is characteristic of the sample. The mobile accessory device may be adapted to receive the sensor output from the mobile analytical device. The remote computing system may be adapted to analyze analytical data using artificial intelligence (AI) and/or machine learning (M-L) to make an authentication determination of the sensor output relative to a. predefined product database. The mobile accessory device may be adapted to upload the sensor output to the remote computing system by a communication network, and the remote computing system may be adapted to download the authentication determination to the mobile accessory device by the communication network.

### CROSS-REFERENCE TO RELATED APPLICATIONS

This application claims priority under 35 U.S.C. § 119 (e) to provisional patent application U.S. Ser. No. 62/646,693, “Click and Bill”, filed Mar. 22, 2018, the contents of which are incorporated herein by reference.

### FIELD OF THE INVENTION

The present invention relates to systems designed to verify the chemical composition or similar characteristic of a product, material, or other sample of interest. In some cases such systems may analyze food samples using Raman spectroscopy or other spectroscopic techniques, or alternative measurement techniques. Aspects of the invention also relate to monetization techniques useful for such systems. The invention also pertains to related methods, systems, and articles.

### BACKGROUND OF THE INVENTION

Various recent reports point to the existence of a global food safety crisis due to widespread counterfeiting and adulteration of food products. For example, a Global Food Safety Initiative Report estimated that food fraud costs the global food industry $30-$40 billion annually. A press release by Europol (Apr. 25, 2017) reported that in the first four months of 2017, Europol and Interpol seized 230 million Euros worth of fake food and beverages. A 2015-2016 annual report of the FSSAI (Indian counterpart to the U.S. Food & Drug Administration) reported that in India during 2015-2016, one out of five food samples analyzed was adulterated.

Sophisticated counterfeiters are quick to imitate successful products, e.g. as illustrated by the widespread and aggressive counterfeiting of Moutai brand liquor in China. Attempts to stop the counterfeiting by the use of various security features on the product packaging, including QR codes, holograms, cap security seals, and UR) tracking, have all failed. Counterfeiters may require only 9-12 months to duplicate such packaging features. Thus, securing the packaging of a product in ways such as this does not verify or guarantee that the contents are authentic,

From a marketing standpoint, food testing is a large and expanding market sector, estimated to grow from $12 billion in 2016 (of which rapid testing constitutes $1.2 billion) to $18.5 billion in 2022 (of which rapid testing will constitute $3.7 billion).

### SUMMARY OF THE INVENTION

A need exists in the industry for new, more effective verification systems for food products or other samples. The application of increasingly effective technologies at different packaging levels can help build an efficient anti-counterfeiting strategy. Relative to product packaging, such as tamper-evident outer pack closure systems including seals, glued flaps, and perforated cartons, authentication technologies such as overt and covert features can provide increased protection. Relative to authentication technologies, traceability technologies such as unique pack identifiers (serial number) combined with online checking systems (end-to-end electronic pedigree) can provide still more protection. Relative to traceability technologies, molecular analysis can provide even more protection.

We have developed a new family of verification systems and methods that employ molecular analysis—in some cases, Raman molecular analysis with—devices that can analyze the product of interest or sample in situ, through the product's outer bottle or container without opening or breaking the seal of the container. Raman spectroscopy is the spectral analysis of light scattered from the sample, the scattered light providing a unique molecular signature based on the (molecular) structure and composition of the sample material. When employed in the disclosed systems, Raman spectroscopy can allow for instant, i.e., nearly instantaneous or at least extremely rapid, product verification of the contents of the container.

The disclosed systems may combine Raman spectroscopy, or other analytical detection devices, with advanced computer technologies such as artificial intelligence (AI), machine learning (M-L), and/or Big Data. The system architecture preferably employs portable, handheld, or compact devices for some of the system functionality, and remote (cloud-based) computer(s) for the advanced computer technologies. In this manner, difficult or time-consuming computational tasks, such as those associated with AI or M-L, can be performed more efficiently on the more powerful remote computer(s), while the raw data to be analyzed, and the output result calculated by the remote computer, can be transferred rapidly between a mobile accessory device such as a smart phone and the remote computer(s).

We therefore disclose herein, among other things, verification systems for testing a sample, comprising a mobile analytical device, a mobile accessory device, and a remote computing system. The mobile analytical device may be adapted to generate a sensor output that is characteristic of the sample. The mobile accessory device may be adapted to receive the sensor output from the mobile analytical device. The remote computing system may be adapted to analyze analytical data using AI and/or M-L to make an authentication determination of the sensor output relative to a predefined product database. The mobile accessory device may be adapted to upload the sensor output to the remote computing system by a communication network, and the remote computing system may be adapted to download the authentication determination to the mobile accessory device by the communication network.

In some cases, the mobile accessory device may be or include a smart phone. In some cases, the mobile analytical device may be or include a compact spectrometer, and the sensor output may be or include a Raman spectrum. In some cases, the remote computing system may be a cloud-based computing system. In some cases, the mobile analytical device may include a button which, when activated by a user, causes the mobile analytical device to analyze the sample, generate the sensor output, and transmit the sensor output to the mobile accessory, device. The activation of the button by the user may further cause the mobile accessory device to upload the sensor output to the remote computing system, and cause the remote computing system to make the authentication determination and download the authentication determination to the mobile accessory device. The activation of the button by the user may further cause the remote computing system to calculate a billing output for the sample analysis performed by the mobile analytical device. Such billing output may be calculated as a function of one, some, or all of (a) whether the authentication determination is positive or negative, or (b) an identity of the sample, or (c) an identity or group of the user.

In some cases, the remote computing system may include a learner module, a monetizer module, a spectra database, and/or a user database.

We also disclose verification systems for testing a sample, comprising a mobile analytical device, a mobile accessory device, and a remote computing system. The mobile analytical device may be adapted to generate a sensor output that is characteristic of the sample. The mobile accessory device may be adapted to receive the sensor output from the mobile analytical device. The remote computing system may be adapted to analyze analytical data using AI and/or M-L to make an authentication determination of the sensor output relative to a predefined product database. Furthermore, the remote computing system may include a billing module that calculates a billing output for a given analysis performed by the mobile analytical device, and the billing output may be calculated as a function of at least one of (a) whether the authentication determination is positive or negative, (b) an identity of the sample, and (c) an identity or group of a user who initiates the given analysis.

We disclose numerous related methods, systems, and articles.

These and other aspects of the present disclosure will be apparent from the detailed description below. In no event, however, should the above summaries be construed as limitations on the claimed subject matter, which subject matter is defined solely by the attached claims, as may be amended during prosecution.

### BRIEF DESCRIPTION OF THE DRAWINGS

The inventive articles, systems, and methods are described in further detail with reference to the accompanying drawings, of which:

FIG. 1 is a perspective view of an exemplary mobile accessory device;

FIG. 2 is a photograph of a setup in which a mobile analytical device is shown proximate a product of interest and in the process of optically sampling such product;

FIG. 3 is a photograph of an exemplary mobile analytical device which includes a physical button that can be activated by a user to cause the analytical device to analyze a given sample, which activation of the button may also initiate a sequence of additional actions including causing the remote computing system to calculate a billing output for the sample analysis performed by the mobile analytical device;

FIG. 4 is a diagram of one embodiment of a network-based verification system as disclosed herein; and

FIG. 5 is a diagram of a managing platform for a company or entity of mobile sensor or detector providers, owners, and/or managers,

In the figures, like reference numerals designate elements.

### DETAILED DESCRIPTION OF ILLUSTRATIVE EMBODIMENTS

We have developed a new family network-based verification systems with new and useful features, and combinations of features, as described and summarized herein. The use of spectroscopy or other measuring techniques and AI-based cloud engines for rapid testing of food or other products enables the systems to provide positive market disruption. For example, Whereas conventional verification systems may perform 2-3 tests/hour, the disclosed systems may perform 20-30 tests/hour, Whereas the test cost (including logistics) may be $150-200/test with conventional systems, it may be as low as $1-2/test for the disclosed systems. Whereas the test machine cost may be $10 k-25 k with conventional systems, it may be as low as $400 with the disclosed systems. Whereas the test machine weight may be 5-15 kg with conventional systems, it may be as low as 250 g with the disclosed systems. Whereas conventional systems may require a controlled environment of 25 degrees C., the disclosed systems may suitable for outdoor temperatures ranging from 0 to 40 degrees C. Whereas the time to receive results may be 1-2 weeks with conventional systems, it may be as low as 1 minute with the disclosed systems. Test sensitivity may be <1% for conventional systems versus 5% for the disclosed systems. The foregoing comparisons are of course generalized and not necessarily applicable to all conventional systems and all of the disclosed systems.

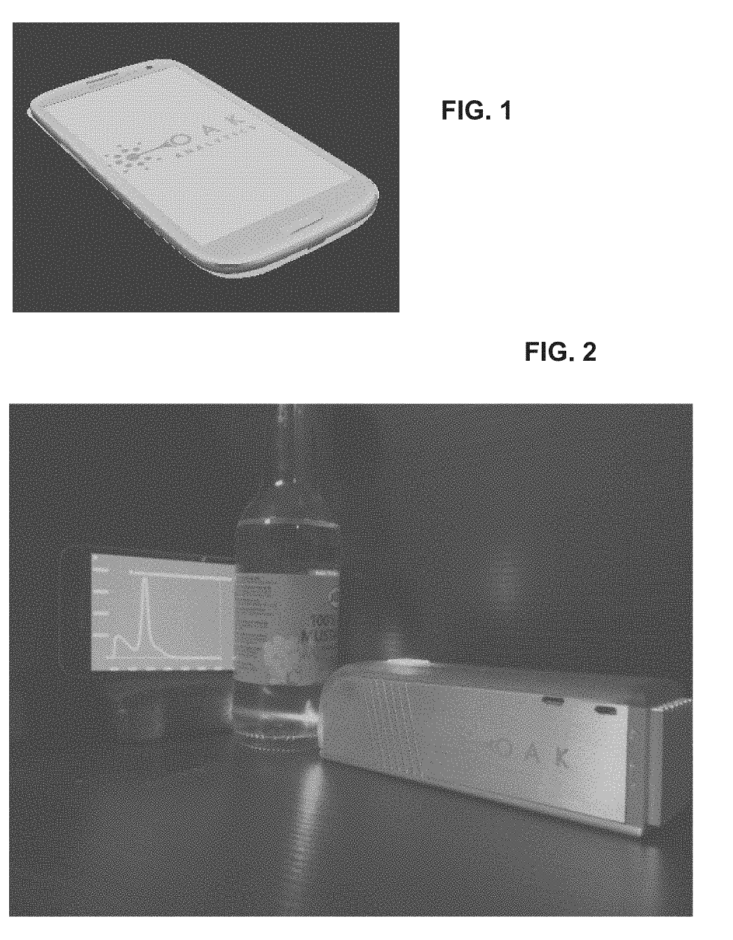

FIG. 1 is a perspective view of an exemplary mobile accessory device that can be used in the disclosed system. In this case, the mobile accessory device is a smart phone. Such a device can communicate and transfer various types of information between a mobile analytical device and a remote computing system. The mobile accessory device may thus for example include a camera such that it can capture an image of a bar code, QR code, or other label information of the sample to be tested so that such labeling information can be uploaded to the remote computing system. The mobile accessory device also of course includes antenna(s) and other components to provide wireless communication to the mobile analytical device, and to the :25 remote computing system e.g., via a conventional cellular network or other suitable communication network.

FIG. 2 is a photograph of a setup in which a mobile analytical device is shown proximate a product of interest, and in the process of optically sampling such product. The mobile analytical device may in some cases be capable of analyzing the product of interest or sample in situ, through the product's outer bottle or container without opening or breaking the seal of the container, as discussed above. In other cases, a mobile analytical device for use in the disclosed systems may not have such remote sampling capabilities. In the background of the photograph of FIG. 2 is the display screen of a smart phone, illustrating a typical sensor output in the form of a spectrum from a tested sample.

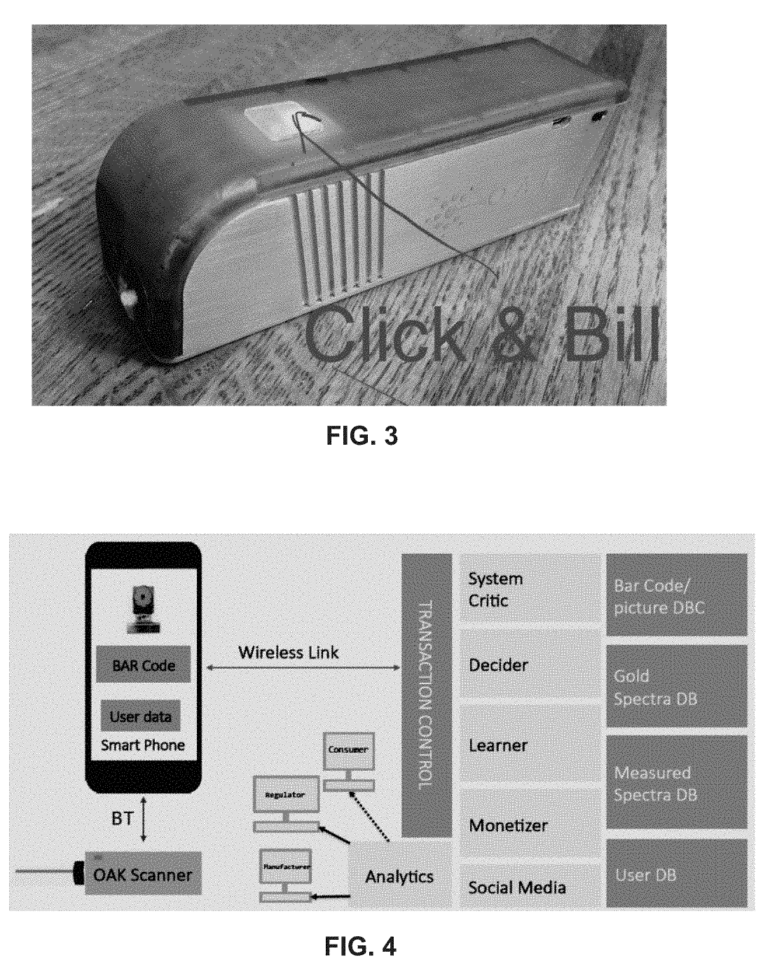

FIG. 3 is a photograph of an exemplary mobile analytical device which includes a physical button that can be activated by a user to cause the analytical device to analyze a given sample, which activation of the button may also initiate a sequence of additional actions including causing the remote computing system to calculate a billing output for the sample analysis performed by the mobile analytical device. Such capability may be referred to as “click and bill”, and it represents a vast change from the way in which conventional analytical testing is conducted and invoiced. With the “click and bill” capability, the user or their administrator or other designated person or entity is invoiced within minutes, rather than days or weeks, after performing the analysis on the sample to verify whether it is authentic or not. The result itself, i.e. the authentication determination which is calculated by the remote computing system, may likewise be delivered to the user or other designated person within minutes of activating the button.

Using the button, a customer may click a scanner to initiate a transaction, whereupon the scanner may capture a spectral signature of the sample of interest, and send such signature to a smart phone/computer. The spectral signature may then be transmitted to the cloud (remote network computer(s)), and cloud-based algorithms may be used to make a determination of authenticity, the result of which may then be reported back to the initiating user. The user or customer will then get billed for the scan, thus completing the transaction.

FIG. 4 is a diagram of one embodiment of a network-based verification system as disclosed herein. Such a system may integrate the Internet of Things (IoT) with AUM-L, and may be designed to support more than 1 million transactions per day. In this figure, we see in schematic form a mobile analytical device (“OAK Scanner”), a mobile accessory device (“Smart Phone”), and a remote computing system, shown as having constituent modules or functions including a Transaction Control function, a System Critic function, a Decider function, a Learner function, a Monetizer function, a Social Media function, an Analytics function, a Bar Code/picture DBC function, a Gold Spectra DB function, a Measured Spectra DB function, and a User DB function. “DB” in this regard of course refers to a database used for the stated purpose. The Analytics function or module may communicate with various users of the system including consumers, regulators, and manufacturers as shown. Information may be conveyed wirelessly such as by the Blue Tooth™ protocol or other wireless protocols between the mobile analytical device and the mobile accessory device, and between the mobile accessory device and the remote computing system.

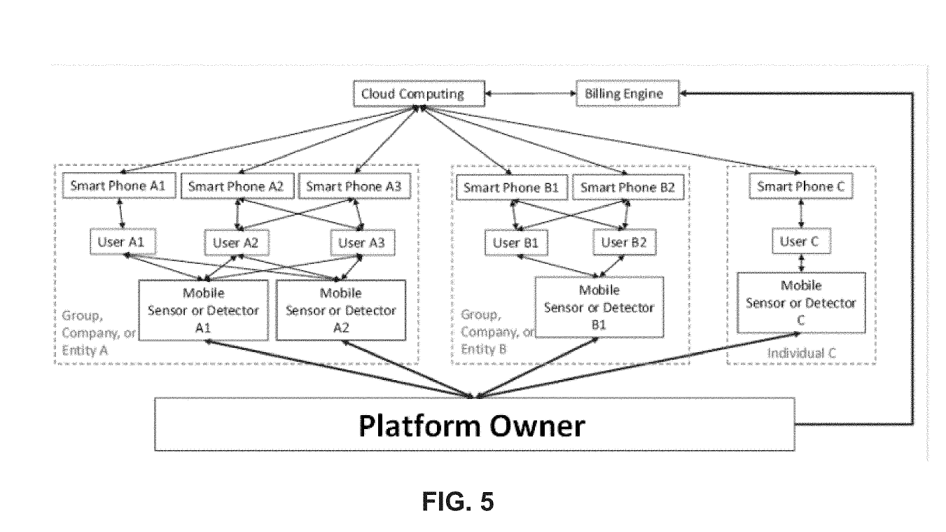

FIG. 5 is a diagram of a managing platform for a company or entity of mobile sensor or detector providers, owners, and/or managers. As shown, users may be categorized according to group characteristics such as those employed with company A versus company B. In some cases users in a given group may exchange or use different smart phones, and may use different mobile analytical devices, which are labeled “Mobile Sensor or Detector” in FIG. 5.

FIG. 5 may thus represent a managing platform for a company or entity of mobile sensor or detector providers, owners, and/or managers. The platform owner may perform one, some, or all of the following functions: form a network of users and mobile sensors or detectors provided by the company or entity; manage users and mobile sensors or detectors provided by the company or entity; collect and analyze mobile sensor or detector data from users; feedback to users judgment or classification based on analyzed result; and bill customers for the service of performing big data analysis. The platform owner may be an analyzer and owner of big data, and. can also be a provider of mobile sensors or detectors, and/or owner of mobile sensors or detectors, and/or manager of mobile sensors or detectors.

In one or many exemplary operational flow charts: (1) a user n may connect a smart phone x via Bluetooth with mobile sensor or detector y; (2) user n (account number n) may use smart phone x to sign into cloud account n; (3) cloud verifies (a) if user n and mobile sensor or detector y are on authorized list, and (b) if user n is authorized to use mobile sensor or detector y; (4) cloud uses billing engine to bill user n; (5) cloud sends signal to smart phone x to authorize use; (6) user n starts to scan using mobile sensor or detector y, and scanned data is stored in smart phone x; (7) smart phone x uploads scanned data to cloud; (8) cloud performs analysis and sends judgment or classification based on analyzed results back to smart phone x.

A network platform may thus facilitate its owner to charge a user on a per use base of using the cloud big data analysis on user's uploaded data, wherein the uploaded data are detected locally by the user using a specific proprietary innovative sensor or detector provided by a company or entity; wherein the big data is established based on the user's uploaded data and the big data is owned by the network platform owner. In some cases, the network platform owner may be also the provider of the specific proprietary innovative sensor or detector.

Unless otherwise indicated, all numbers expressing quantities, measured properties, and so forth used in the specification and claims are to be understood as being modified by the term “about”. Accordingly, unless indicated to the contrary, the numerical parameters set forth in the specification and claims are approximations that can vary depending on the desired properties sought to be obtained by those skilled in the art utilizing the teachings of the present application. Not to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, to the extent any numerical values are set forth in specific examples described herein, they are reported as precisely as reasonably possible. Any numerical value, however, may well contain errors associated with testing or measurement limitations.

Various modifications and alterations of this invention will be apparent to those skilled in the art without departing from the spirit and scope of this invention, which is not limited to the illustrative embodiments set forth herein. The reader should assume that features of one disclosed embodiment can also be applied to all other disclosed embodiments unless otherwise indicated. All U.S. patents, patent application publications, and other patent and non-patent documents referred to herein are incorporated by reference, to the extent they do not contradict the thregoing disclosure.

What is claimed is:

1. A verification system for testing a sample, comprising:
a mobile analytical device adapted to generate a sensor output that is characteristic of the sample;
a mobile accessory device adapted to receive the sensor output from the mobile analytical device; and
a remote computing system adapted to analyze analytical data using AI and/or M-L to make an authentication determination of the sensor output relative to a predefined product database;
wherein the mobile accessory device is adapted to upload the sensor output to the remote computing system by a communication network, and the remote computing system is adapted to download the authentication determination to the mobile accessory device by the communication network.

2. The system of claim 1, wherein the mobile accessory device includes a smart phone.

3. The system of claim 1, wherein the mobile analytical device includes a compact spectrometer, and wherein the sensor output includes a Raman spectrum.

4. The system of claim 1, wherein the remote computing system is a cloud-based computing system.

5. The system of claim 1, wherein the mobile analytical device includes a button which, when activated by a user, causes the mobile analytical device to analyze the sample, generate the sensor output, and transmit the sensor output to the mobile accessory device.

6. The system of claim 5, wherein the activation of the button by the user further causes the mobile accessory device to upload the sensor output to the remote computing system, and causes the remote computing system to make the authentication determination and download the authentication determination to the mobile accessory device.

7. The system of claim 6, wherein the activation of the button by the user further causes the remote computing system to calculate a billing output for the sample analysis performed by the mobile analytical device.

8. The system of claim 7, wherein the billing output is calculated as a function of whether the authentication determination is positive or negative.

9. The system of claim 7, wherein the billing output is calculated as a function of an identity of the sample.

10. The system of claim 7, Wherein the billing output is calculated as a function of an identity or group of the user.

11. The system of claim 1, wherein the remote computing system includes a learner module.

12. The system of claim 1, wherein the remote computing system includes a monetizes module.

13. The system of claim 1, wherein the remote computing system includes a spectra database.

14. The system of claim 1, wherein the remote computing system includes a user database.

15. A verification system for testing a sample, comprising:
a mobile analytical device adapted to generate a sensor output that is characteristic of the sample;
a mobile accessory device adapted to receive the sensor output from the mobile analytical device; and
a remote computing system adapted to analyze analytical data using AI and/or M-L to make an authentication determination of the sensor output relative to a predefined product database;
wherein the remote computing system includes a billing module that calculates a billing output for a given analysis performed by the mobile analytical device, and Wherein the billing output is calculated as a function of at least one of (a) whether the authentication determination is positive or negative, (b) an identity of the sample, and (c) an identity or group of a user who initiates the given analysis.

16. The verification system of claim 15, wherein the billing output is calculated as a function of at least two of (a) whether the authentication determination is positive or negative, (b) the identity of the sample, and (c) the identity or group of the user who initiates the given analysis.
